(19) United States
(12) Plant Patent Application Publication
(10) Pub. No.: US 2004/0098772 P1
(43) Pub. Date: May 20, 2004

(54) GRAPEVINE PLANT NAMED '14-48-258'

(76) Inventor: Timothy F. Bourne, Visalia, CA (US)

Correspondence Address: JONDLE & ASSOCIATES P.C., 9085 EAST MINERAL CIRCLE, SUITE 200, CENTENNIAL, CO, 80112, US

(21) Appl. No.: 10/295,253

(22) Filed: Nov. 15, 2002

(51) Int. Cl. A01H 5/00

(52) U.S. Cl. PLT/205

(57) ABSTRACT

This new seeded variety of table grapevine ‘14-48-258’ is new and different because of its firm texture, uniform clusters and large berry size. Its clusters are well-filled, but loose; its large berries are sweet and mild. The vigorously growing vines are extremely productive when spur pruned.

### GENUS AND SPECIES

Vitis vinifera

### BACKGROUND AND SUMMARY OF THE INVENTION

The new table grapevine cultivar ‘14-48-258’ is of Vitis vinifera parentage. The goal in the development of this new table grape was to provide an alternative to the widely grown cultivar ‘Alphonse Lavalle’ (unpatented).

The seeded, female parent of the new variety is an un-named black table grape of uncertain parentage with large berry size and high fruitfulness. The seedless, male parent of the new cultivar is the variety ‘Fantasy Seedless’ (unpatented). The hybridization resulting in the new cultivar was made in 1993 near Delano, Calif. Seed from the cross was harvested, stratified and sown in a greenhouse in January 1994. The resulting 47 seedlings were planted in a field in the same area in March of the same year. In 1997, fruit was seen on the vine for the first time and it was selected for further trial. Vines were propagated from the original seedling in the spring of 1998 with own-rooted and grafted vines planted in the original trial. Those vines and subsequent propagules have all been true to type.

### COMPARISON WITH PARENTAL CULTIVAR

The new cultivar differs from its ancestor, ‘Alphonse Lavalle’ by having a much more well-filled cluster. The berries of ‘14-48-258’ are about 2 g. larger than ‘Alphonse Lavalle’ on average and have flesh which is firmer than that of the latter cultivar. Additionally, the berry shape of ‘14-48-258’ is generally elliptic to obovate with clearly defined lobing on the stigmatic end of the berry, whereas ‘Alphonse Lavalle’ has nearly spherical berries.

### DESCRIPTION OF THE FIGURES

The accompanying drawing illustrates the following:

FIG. 1: Cane, leaf, and fruit clusters.

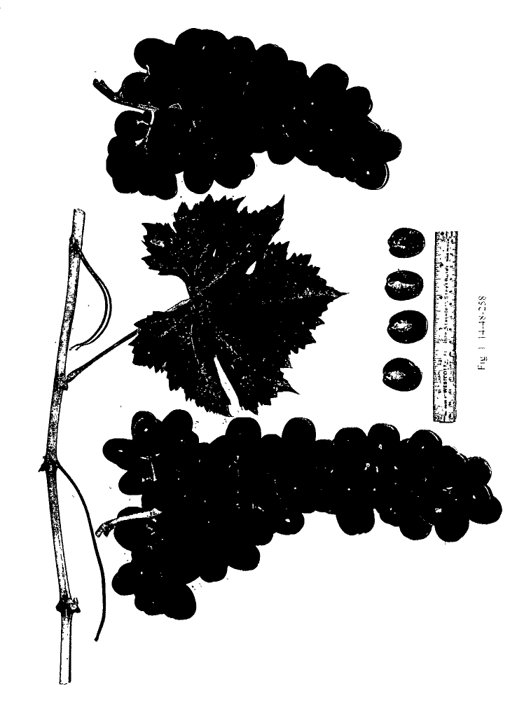

### DETAILED BOTANICAL DESCRIPTION OF THE INVENTION

The following description of grapevine ‘14-48-258’ contains references to color names taken from the Munsell Color Chart for Plant Tissues, published by Munsell Color, New Windsor, N.Y. Descriptors used herein conform to those set forth by the International Board for Plant Genetic Resources Institute Grape Descriptors (Vitis spp.) of 1983 and/or 1997 which were developed in collaboration with the Office International de la Vigne et du Vin (OIV) and the International Union for the Protection of New Varieties of Plants (UPOV).

Descriptions of the new invention apply to vines of ‘14-48-258’ grown on ‘Freedom’ rootstock at a density of 1,537 vines per hectare grown in Kern County, California in 2001. These vines were in their first year of full production having been planted in 1998. These descriptions are believed to apply generally to the new variety grown under similar circumstances elsewhere:

### VINE

General:

Size.—Large.

Vigor.—High.

Density of foliage.—Dense.

Productivity.—Very productive when spur pruned, up to 64,157 kg./hectare.

Trunk circumference.—15.4 cm. at 1 meter height.

Rootstock.—Freedom.

Trunk:

Shape.—Medium.

Straps.—Long, split.

Surface texture.—Shaggy.

Inner bark color.—5YR 4/6.

### LEAVES

Mature leaves:

Average blade length.—16.0 cm.

Average blade width.—22.5 cm.

Size of blade.—Large.

Shape.—Pentagonal.

Anthocyanin coloration of main veins on the upper side of the blade.—Strong.

Mature leaf profile.—Wavy.

Blistering surface of blade upper surface.—Absent.

Leaf blade tip.—In plane of the leaf.

Undulation of margin.—Slight.

Apex.—Acuminate.

Thickness.—Medium.

Undulation of blade between main and lateral veins.—Strong.

Shape of teeth.—Conical, both sides convex.

Length of teeth.—3-10 mm.

Ratio length/width of teeth.—About 1:1.

General shape of petiole sinus.—Variable — open (ovate) to closed.

Tooth at petiole sinus.—Absent.

Petiole sinus limited by veins.—Absent.

Shape of upper lateral sinus.—Closed.

Prostrate hairs between veins on lower surface of blade.—Absent.

Erect hairs between veins on lower surface of blade.—Absent.

Prostrate hairs on main veins on lower surface of blade.—Absent.

Density of erect hairs on main veins on lower surface of blade.—Sparse, confined to junction of veins.

Prostrate hairs on main veins on upper surface of blade.—Absent.

Autumn coloration.—Green until frost: 7.5GY 4/4 to 3/4.

Upper surface:

Color.—7.5 GY 4/4 to 3/4.

Surface texture.—Smooth.

Surface appearance.—Dull.

Goffering of blade.—Absent.

Lower surface:

Color.—7.5GY 4/6.

Anthocyanin coloration of main veins on lower leaf surface.—Present.

Glossiness.—Low.

Pubescence.—Absent.

Surface texture.—Smooth.

Surface appearance.—Dull.

Petiole:

Length of petiole.—16.2 cm.

Length of petiole compared to middle vein.—Approximately equal.

Density of prostrate hairs on petiole.—Absent.

Density of erect hairs on petiole.—Absent.

Shape of base of petiole sinus.—Mostly closed, some ½ open, ovate.

Woody shoot:

Shape.—Stocky.

Internode length.—About 9.5 cm.

Width at node.—About 2.0 cm.

Cross section.—Circular.

Surface.—Smooth.

Main color.—5YR 5/6.

Lenticels.—Inconspicuous.

Erect hairs on nodes.—Absent.

Erect hairs on internodes.—Absent.

Growth of axillary shoots.—Prolific.

Buds:

Shape.—Conical.

Cane bud fruitfulness.—Basal buds fruitful, 1-2 clusters per shoot.

### FLOWERS

General:

Flower sex.—Perfect.

Length of first inflorescence.—23.1 cm.

Position of first flowering nodes.—3^{rd} to 4^{th}.

Number of inflorescences per shoot.—1 or 2.

Date of full bloom.—May 14, 2001.

Time of bloom.—Average for grapes growing in the area around McFarland, Calif.

### FRUIT

General:

Ripening period.—Late, mid-season, about 3 weeks after ‘Thompson Seedless’ at Delano, Calif.

Date of ripening.—About September 1st.

Use.—Fresh market.

Keeping quality.—Very good.

Resistance.—Insects: typical of Vitis vinifera. Diseases: typical of Vitis vinifera.

Shipping quality.—Good.

Date of first harvest.—Sep. 1, 2001.

Solids-sugar.—About 20 brix at full maturity.

Refractometer test.—19.0 brix.

Cluster:

Bunch size.—Medium.

Bunch length (peduncle excluded).—About 30.0 cm.

Bunch width.—About 9.0 cm.

Bunch weight (natural).—1,392 g.

Bunch weight (gibberellic acid treated).—1,069 g.

Bunch density.—Very dense.

Number of berries.—97.3.

Form.—Conical.

Peduncle:

Length of peduncle.—2.7 cm.

Lignification of peduncle.—Medium, upper 1 cm.

Color.—7.5YR 5/8.

Berry:

Size.—Large.

Uniformity of size.—Uniform.

Berry weight.—12.8 g.

Shape.—Obovate to elliptic.

Presence of seeds.—2-4 per berry, averaging 43 mg each.

Cross section.—Circular.

Dimensions.—Longitudinal axis about 3.0 cm.; horizontal axis about 2.2 cm.

Skin color (without bloom).—Dark red-black, 5RP 3/2.

Coloration of flesh.—Clear.

Juiciness of flesh.—Very juicy.

Berry firmness.—Very firm.

Particular flavor.—Neutral, typical vinifera.

Bloom (cuticular wax).—Strong.

Pedicel length.—7 mm.

Berry separation from pedicel.—Moderate.

Skin:

Thickness.—Medium.

Texture.—Tender.

Reticulation.—Absent.

Roughness.—Absent.

Tenacity.—Tenacious to flesh.

Tendency to crack.—Resistant.

What is claimed is:

1. The new and distinct variety of TABLE GRAPE ‘14-48-258’ herein described and illustrated and identified by the characteristics enumerated above.
